(19) United States
(12) Patent Application Publication
(10) Pub. No.: US 2006/0020172 A1
(43) Pub. Date: Jan. 26, 2006

(54) OCT laryngoscope

(75) Inventors: Kathrin Luerssen, Hannover (DE); Holger Lubatschowski, Gehrden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: Rowiak GmbH.

(21) Appl. No.: 11/185,794

(22) Filed: Jul. 21, 2005

(30) Foreign Application Priority Data
Jul. 21, 2004 (DE) 102004035269.0-35

(51) Int. Cl. A61B 5/05; A61B 1/267; A61B 1/06

(52) U.S. Cl. 600/188; 600/109; 600/160; 600/425

(57) ABSTRACT

The invention relates to a laryngoscope with a light guiding portion for insertion into a patient's oral cavity and an observation device for diagnostics with visible light, comprising an illumination beam path for illuminating an examination area with a visible observation beam, and an imaging beam path for guiding the observation beam reflected from the examination area. Such known laryngoscopes permit diagnostics of pathological changes in deeper tissue layers. The laryngoscope of the invention enables this by means of an OCT device for diagnostics using optical coherence tomography, comprising an OCT illumination beam path for illuminating an examination area with the coherent beam, and an OCT imaging beam for guiding the coherent beam reflected from the examination area to an OCT module for generating an image of tissue layers in the examination area.

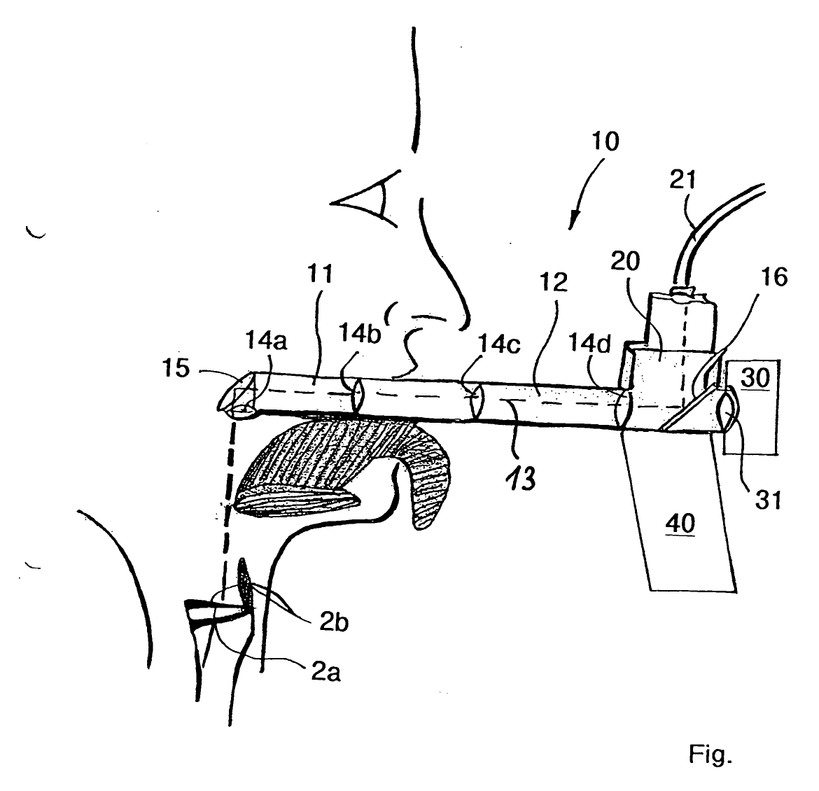

### FIELD OF THE INVENTION

The invention relates to a laryngoscope comprising a light guiding portion for insertion into a patient's oral cavity and an observation device for diagnostics using visible light, an illumination beam path for illuminating an examination area with a visible observation beam, an imaging beam path for guiding the observation beam reflected from the examination area, a first optical aperture through which the observation beam reflected from the examination area enters the laryngoscope, and a second optical aperture for guiding the reflected observation beam to an examiner.

### BACKGROUND OF THE TECHNOLOGY

Laryngoscopes of the aforementioned kind are used for optical diagnostics in ear, nose and throat medicine, and are used in particular for imaging diagnostics of the vocal folds. For this purpose, the laryngoscope is inserted into the patient's oral cavity and advanced so far in the direction of the pharyngeal cavity that it is possible to observe the vocal folds when the dorsum of the tongue is lowered by pulling the tongue outwards from the mouth and downwards. A laryngoscope usually has a deflection device at the end which is inserted into the oral cavity, in order to bend the optical observation axis by about 70-90 degrees.

Known laryngoscopes, such as the laryngoscope known from EP 0 901 772 A1, for example, thus permit real-time observation of the vocal folds. However, many pathological changes of the vocal folds cannot be reliably diagnosed, or diagnosed at all, using the static observation of the vocal folds enabled by such laryngoscopes.

It is medical routine in the ENT field to use a stroboscopic light to illuminate the patient's vocal folds during phonation. Matching the strobe frequency to the resonant frequency of the vocal folds for the respective phonation pitch enables the dynamic behavior of the vocal folds to be observed. A phase shift between the strobe frequency and the resonant frequency of the vocal folds enables the resonance behavior of the vocal folds to be observed in slower motion.

However, even when using this diagnostic method, it is often the case that pathological changes deep in the tissue structure of the vocal folds cannot be diagnosed or diagnosed reliably.

### SUMMARY OF THE INVENTION

The object of the present invention is to provide a laryngoscope for improved diagnosis of pathological changes, in particular pathological changes of the vocal folds.

In a laryngoscope of the kind initially specified, this object is achieved with an OCT device for diagnostics using optical coherence tomography, comprising an OCT illumination beam path for illuminating an examination area with a coherent beam, and an OCT imaging beam for guiding the coherent beam reflected from the examination area to an OCT module for generating an image of tissue layers in the examination area.

The laryngoscope developed in this manner allows the tissue observed with the observation device to be imaged with optical coherence tomography (OCT). A measurement beam of optically coherent light is guided onto a measurement point and reflected by the tissue layers from different depths. Due to the differences in path lengths into the separate tissue layers at greater or smaller depths, it is possible to allocate the backscattered light to the respective tissue layers by measuring the interference. The depth of penetration into the tissue depends on the wavelength of radiation used; infrared or near-infrared radiation with a penetration depth of about 3 mm into the tissue is typically used.

In order that larger areas of tissue can also be imaged with optical coherence tomography, the OCT measurement beam can be guided over such a larger surface to produce a scanned image.

The laryngoscope developed in this manner enables deeper tissue layers in the vocal folds to be imaged in a form that is very helpful for diagnosis, and therefore can also show pathological changes in these deeper tissue layers.

The separate beam paths of the laryngoscope according to the invention may take the form of fiber optics, in particular.

The OCT device disposed on the laryngoscope allows an OCT detector and an OCT radiation source disposed on or at a distance from the laryngoscope, and which may also include an OCT scanner, to be connected to the laryngoscope. The illumination beam path and/or the imaging beam path of the OCT device can be guided via guiding means from the laryngoscope to the OCT detector and the OCT scanner, or the OCT scanner and OCT detector can be mounted on the laryngoscope itself.

### BRIEF DESCRIPTION OF THE FIGURE

The FIGURE shows a patient examined with a laryngoscope of the invention.

### DETAILED DESCRIPTION

In a first advantageous embodiment of the invention, the illumination beam path and the imaging beam path run coaxially at least in that portion of the laryngoscope which is insertable into the oral cavity. In this way, the illumination beam source can be coupled into the imaging beam path via a semi-permeable mirror, with the result that the observation device can be configured with a single beam path that can be accommodated within a single fiber optic.

It is also advantageous when the OCT illumination beam path and the OCT imaging beam path run coaxially at least in that portion of the laryngoscope which is insertable into the oral cavity. In this way, the OCT device of the laryngoscope can be configured with a single beam path that can be accommodated within a single fiber optic.

It is also advantageous when at least one beam path of the observation device and at least one beam path of the OCT device run coaxially at least in that portion of the laryngoscope which is insertable into the oral cavity. This embodiment permits a more compact design on the whole for the laryngoscope according to the invention. In particular, it is advantageous when all four beam paths of the laryngoscope run coaxially such that all the required beam paths can be combined along a single optical axis, in particular in a single fiber optic. This allows a particularly compact design of that portion of the laryngoscope which is inserted into the oral cavity, and hence only slight discomfort for the patient during the examination.

The laryngoscope may be further developed by a means for combining and splitting at least one beam path of the observation device and at least one beam path of the OCT device. This development enables the beam paths to be combined and/or split, for example in that portion of the laryngoscope situated outside the oral cavity, thus enabling a thinner structure for the portion inserted into the oral cavity, on the one hand, as well as simpler structural designs for the observation device and for the OCT device outside the oral cavity.

It is particularly advantageous in this regard when the means for combining and splitting the beam paths comprises a pleochroitic, in particular, a dichroitic beam splitter. A beam splitter of this kind is an optical component with optical properties that vary according to the wavelength of the radiation passing through the component. In particular, such an optical component can be configured so that radiation of a certain wavelength is reflected at a specific angle, while radiation of a different wavelength is not reflected. In the case of the laryngoscope according to the invention, a dichroitic beam splitter, i.e. one that responds differently to two different wavelengths or wavelength ranges, is particularly advantageous for splitting the OCT radiation, which is typically in the non-visible infrared range, from the visible range beams of the observation device.

It is also advantageous when the OCT device comprises an OCT module attachable to the laryngoscope, and said module has an OCT scanner and an OCT detector. A laryngoscope of the latter kind is particularly easy to handle and allows an examiner to easily target, focus and handle the laryngoscope.

It is also advantageous when the operating distance of the OCT device is approximately equal to the operating distance of the observation device and is equal, in particular, to the distance between the first optical aperture of the laryngoscope inserted into a patient's oral cavity and the patient's vocal folds. When conducting examinations in the pharyngeal region, it is generally problematic to insert diagnostic instruments until they are close to the area being examined, because this often triggers the patient's swallowing reflex or, especially, the gag reflex. It is therefore particularly advantageous to be able to observe the examined area from a greater distance. For example, it is particularly advantageous when the end of the laryngoscope to be inserted into the oral cavity need only be advanced to the start of the pharyngeal cavity in order to enable the vocal folds to be observed from a distance of approximately 4 to 8 cm. The operating distance of the laryngoscope is therefore advantageously designed for this distance.

In another development of the laryngoscope according to the invention, the depth of field or depth of focus range of the observation device is substantially equal to the operating range of the OCT device.

In this context, the operating distance is understood to be the distance between the first optical opening and the area being examined. The operating range is understood in this context to be the range of tolerance within which the operating distance can be varied without significantly reducing the quality of the image obtained. Therefore, the range limits to be set for the operating distance are calculated as the operating distance plus/minus half the operating range.

Currently known optical coherence tomographs can typically only operate in a range of 2-5 mm. This means that, to obtain precise images with the OCT, it is necessary to routinely adjust the operating distance, i.e. the distance between the first optical opening and the area being examined, with a precision of approximately 2-5 mm.

The operating distance can be precisely adjusted if, in particular, the examiner adjusts optical focusing means to focus the image obtained with the observation means, wherein said focusing means acts in equal measure on the OCT radiation and thus modifies the operating distance of the OCT device. An alternative or addition to this method is to leave the focusing and hence the operating distance unchanged, and, by moving the laryngoscope, to adjust the distance between the examination area and the laryngoscope until the image obtained with the observation device is sharply focused and the correct operating distance thus achieved.

If the depth of field of the observation device is selected so that it is approximately the same as the operating range of the OCT device, bringing the area being examined into sharp focus with the observation device simultaneously ensures at all times that the correct operating distance has been set for the OCT device.

The latter embodiment is particularly advantageous when the beam paths of the observation device and the OCT device run coaxially and therefore strike the examination area from a common optical opening, or are guided from the examination area through said opening into the laryngoscope. In such a case, the distance between the first optical opening and the area being examined, i.e. the operating distance, can be set by focusing and/or moving the laryngoscope, thus ensuring that the distance does not go beyond the operating range of the OCT device.

Another development of the laryngoscope according to the invention is characterized by means for influencing the depth of field of the observation device, in particular for matching the depth of focus to the operating range of the OCT device. In particular, such means enables the depth of field of a laryngoscope's optics to be reduced in order to match it to the small operating range of the OCT device.

It is particularly advantageous in this context when the means for influencing the depth of field can be deactivated. One way of deactivating said means is to move optical components out of or into the beam path. This development of the invention enables an initially conventional examination to be carried out with the laryngoscope and with deactivated adjustment means, before activating the adjustment means, then performing the necessary adjustments and focusing so that diagnostics can be carried out with the OCT device.

In the two embodiments described in the foregoing, it is particularly advantageous when the means for influencing the depth of field includes an aperture in the imaging beam path and the size of said aperture is selected such that the depth of field of the observation device is substantially equal to the operating range of the OCT device, and in particular does not exceed the operating range of the OCT device. Such an aperture can comprise a shutter, for example, with an aperture diameter that is selected according to the desired depth of field. A shutter of this kind can then be pivoted away from the beam path if so required, or can be replaced by a smaller shutter in order to cancel its effect on the depth of field.

Another development of the aforementioned embodiments with means for influencing the depth of field includes means for increasing the total magnification of the observation device, said means being selected such that the depth of field of the observation device is substantially equal to the operating range of the OCT device, and in particular does not exceed the operating range of the OCT device. This development of the invention enables not only reliable adjustment to the operating range of the OCT device, as described above, but also very precise adjustment of said operating range due to the fact that a magnified image of the area being examined is made possible. Such means for influencing the depth of field can be in the form, for example, of one or two optical lens that can also be moved out of the beam path when it is necessary to deactivate this influence.

It is particularly advantageous in this context when the means for increasing the total magnification is configured for continuous increase in total magnification. Zoom optics of this kind allow the total magnification to be adjusted to the different examination situation and anatomy in each case.

Another advantageous development of the laryngoscope according to the invention comprises means for increasing the depth of field and/or the visual field of the observation device. By providing easier orientation and an improvement on conventional diagnostics, this development enables improved observation of the area being examined, with facilitated focusing and a greater observation range. Such additional means for increasing the depth of field can be configured, for example, in the form of a smaller shutter.

It is particularly advantageous here when the means for increasing the depth of field and/or the visual field of the observation device can be deactivated. In this way, after basic orientation and conventional examination using the laryngoscope, the means for increasing the depth of field can be deactivated, for example by moving the respective means out of the beam path and subsequently performing an examination using the OCT device.

Another development of the invention is characterized by means for generating a visible pilot beam directed onto the area being examined by the OCT device. Since the OCT radiation is typically in the non-visible range, it is not possible for an examiner to identify the position on the examination area that is being subjected to OCT diagnostics. It is therefore advantageous when a pilot beam is disposed coaxially to the OCT beam, thus marking the OCT measurement point. The pilot beam can also be configured in such a way that it marks the area being scanned by the OCT measurement beam. This can be effected by lateral illumination of the OCT measurement area, or by framing this OCT measurement area, for example.

It is particularly advantageous when the second optical aperture co-operates with an image detection device, in particular a CCD camera. This enables the images recorded by the observation device to be saved, and also enables these images to be displayed at a location remote from the laryngoscope.

To this end, it is also advantageous when an image rendition device is available for displaying the image recorded by the image detection equipment. Such an image rendition device can be provided, for example, in the form of a screen or projector, and allows a plurality of observers to view the images obtained with the observation device.

It is particularly advantageous when the second optical aperture is configured as an eyepiece to enable the examination area to be viewed directly by the eye of an examiner. This enables the laryngoscope according to the invention to be used in a conventional manner, and therefore allows those examiners in particular who have many years of practical experience with conventional laryngoscopes to make easy and supplementary use of the additional diagnostics that can be achieved with the laryngoscope of the invention and the OCT images it produces.

Finally, it is advantageous when the illumination beam path can be combined with a stroboscope to illuminate the area being examined. By this means, the laryngoscope according to the invention enables not only laryngostroboscopic diagnostics of the known kind to be performed, but also and additionally the conventional static observation and OCT imaging of the vocal folds in the manner according to the invention.

A preferred embodiment shall now be described with reference to the attached FIGURE. The FIGURE shows a partly cutaway side view of the laryngoscope according to the invention, with the laryngoscope inserted into a patient's oral cavity.

The FIGURE shows a patient 1, with vocal folds 2a, b, and a laryngoscope 10 inserted into the oral cavity.

Laryngoscope 10 has a first portion 11 that is insertable into the oral cavity, said first portion having a substantially tubular form. Said first portion 11 extends outside the oral cavity into a second tubular portion 12.

An optical axis 13 runs inside portions 11, 12. The illumination beam path and the imaging beam path of the observation device, and the OCT illumination beam path and the OCT imaging beam path of the OCT device run coaxially along said optical axis. These four beam paths are guided spaced apart from each other through a plurality of optical components 14a-d disposed along optical axis 13.

A beam deflector 15, for example a prism, is disposed at the end of portion 11 which is inserted into the oral cavity and deflects the beam by approximately 70 to 90 degrees.

A dichroitic beam splitter 16 in the form of a mirror is disposed in the beam path and along optical axis 13 at the end of the laryngoscope which is located outside the patient and deflects the OCT radiation by 90 degrees while allowing visible spectrum radiation to pass through without deflection. By this means, the OCT illumination beam path and the OCT imaging beam path are deflected by 90 degrees and guided into an OCT module 20.

OCT module 20 is connected by a fiber optic cable 21 to an OCT radiation source and an OCT detector (not shown). Inside OCT module 20 there is an OCT scanner for deflection and for scanning an OCT examination area, as well as telescope optics for adjusting the OCT radiation to the optics of the imaging beam path in portions 11, 12, along which the OCT radiation reflected by vocal folds 2a, b travels.

Visible range light passing through the dichroitic beam splitter 16 is guided by an ocular lens 31 to a CCD camera 30 and allows the vocal folds 2a, b to be imaged on a screen (not shown). CCD camera 30 is detachably mounted on the laryngoscope so that it is also possible for the examiner to observe vocal folds 2a, b directly through eyepiece 31.

A handle 40 enabling the examiner to handle and align the laryngoscope with ease is disposed opposite OCT module 20 with respect to optical axis 13.

1. Laryngoscope, comprising
a light guiding portion (11) for insertion into a patient's oral cavity and
an observation device (14a-d, 31, 30) for diagnostics with visible light, comprising
an illumination beam path (13) for illuminating an examination area (2a, b) with a visible observation beam,
an imaging beam path (13) for guiding the observation beam reflected from the examination area,
a first optical aperture (14a) through which the observation beam reflected from the examination area enters the laryngoscope, and
a second optical aperture (31) for guiding the reflected observation beam to a examiner,
characterized by an OCT device (14a-d, 16) for diagnostics using optical coherence tomography, comprising
an OCT illumination beam path (13) for illuminating an examination area with the coherent radiation, and
an OCT imaging beam path (13) for guiding the coherent radiation reflected from the examination area to an OCT module (20) for generating a tissue layer image of the examination area.

2. Laryngoscope according to claim 1,
wherein the illumination beam path (13) and the imaging beam path (13) run coaxially at least in that portion (11) of the laryngoscope that is insertable into the oral cavity.

3. Laryngoscope according to claim 1,
wherein the OCT illumination beam path (13) and the OCT imaging beam path (13) run co-axially at least in that portion (11) of the laryngoscope that is insertable into the oral cavity.

4. Laryngoscope according to claim 1,
wherein at least one beam path (13) of the observation device and at least one beam path (13) of the OCT device run coaxially at least in that portion (11) of the laryngoscope that is insertable into the oral cavity.

5. Laryngoscope according to claim 1,
further comprising means (16) for combining and splitting at least one beam path (13) of the observation device and at least one beam path (13) of the OCT device.

6. Laryngoscope according to claim 5,
wherein said means for combining and splitting the beam paths comprises a pleochroitic beam splitter (16).

7. Laryngoscope according to claim 1,
wherein the OCT device further comprises an OCT module (30) attachable to the laryngoscope, said module having an OCT scanner and telescope optics.

8. Laryngoscope according to claim 1,
wherein the operating distance of the OCT device is approximately equal to the operating distance of the observation device.

9. Laryngoscope according to claim 1,
wherein the depth of field of the observation device is substantially equal to the operating range of the OCT device.

10. Laryngoscope according to claim 1,
further comprising means (14a-d) for influencing the depth of field of the observation device.

11. Laryngoscope according to claim 9,
wherein said means for influencing the depth of field can be deactivated.

12. Laryngoscope according to claim 9,
wherein said means for influencing the depth of field includes an aperture in the imaging beam path that is selected with such a size that the depth of field of the observation device is substantially equal to the operating range of the OCT device.

13. Laryngoscope according to claim 9,
wherein said means for influencing the depth of field includes means (31) for increasing the total magnification of the observation device, said means being selected such that the depth of field of the observation device is substantially equal to the operating range of the OCT device and does not exceed the operating range of the OCT device.

14. Laryngoscope according to claim 13,
wherein said means for increasing the total magnification is configured for continuous increase in total magnification.

15. Laryngoscope according to claim 1,
further comprising means (31) for increasing the depth of field and/or the visual field of the observation device.

16. Laryngoscope according to claim 15,
wherein said means for increasing the depth of field and/or the visual field of the observation device can be deactivated.

17. Laryngoscope according to claim 1,
further comprising means for generating a visible pilot beam directed onto the area being examined by the OCT device.

18. Laryngoscope according to claim 1,
wherein the second optical aperture (31) co-operates with an image detection device (30).

19. Laryngoscope according to claim 18,
further comprising an image rendition device for rendering the image recorded by the image detection device.

20. Laryngoscope according to claim 1,
wherein the second optical aperture (31) is configured as an eyepiece to enable the examination area to be viewed directly by the eye of an examiner.

21. Laryngoscope according to claim 1,
wherein the illumination beam path (13) can be combined with a stroboscope to illuminate the area being examined.
